SEQUENCE LISTING (11 sequences)

SEQ ID NO: 1 - protein, 233 aa, synthetic construct
  source 1..233: note = Variant nhhB
MNGVYDVGGTDGLGPINRPADEPVFRAEWEKVAFAMFPATFRAGFMGLDEFRFGIEQMNPAEYLESPYYWHWIRTYIHHGVRTGKIDLEELERRTQYYRENPDAPLPDHEQKPELIEFVNQAVYGGLPASREVDRPPKFKEGDVVRFSTASPKGHARRARYVRGKTGTVVKHHGAYIYPDTAGNGLGECPEHLYTVRFTEQELWGPEGDPNSSVYYDCWEPYIELVDTKAAAA

SEQ ID NO: 2 - protein, 205 aa, synthetic construct
  source 1..205: note = Variant nhhA
MTENITRKSDEEIQKEITVRVKALESMLIEQGILTTSMIDRMAEIYENEVGPHLGAKVVVKAWTDPEFKKRLLADGTEACKELGIGGLQGEDMMWVENTDEVHHVVVCTLCSCYPWPVLGLPPNWYKEPQYRSRVVREPRQLLKEEFGFEVPPSKEIKVWDSSSEMRFVVLPQRPAGTDGWSEEELATLVTRESMIGVEPAKAVA

SEQ ID NO: 3 - protein, 144 aa, synthetic construct
  source 1..144: note = nhhG activator protein
MSAEAKVRLKHCPTAEDRAAADALLAQLPGGDRALDRGFDEPWQLRAFALAVAACRAGRFEWKQLQQALISSIGEWERTHDLDDPSWSYYEHFVAALESVLGEEGIVEPEALDERTAEVLANPPNKDHHGPHLEPVAVHPAVRS

SEQ ID NO: 4 - DNA, 702 bp, synthetic construct
  source 1..702: mol_type = other DNA; note = Variant DSM 43832 nhhB
atgaacggcgtttacgacgttggcggcaccgacggcctgggtccaatcaaccgcccagcagacgagccagttttccgcgctgagtgggagaaggttgcattcgctatgttcccagcaaccttccgcgctggcttcatgggcctggacgagttccgcttcggcatcgagcagatgaacccagcagagtacctggagtccccatactactggcactggatccgcacctacatccaccacggcgttcgcaccggcaagatcgacctggaggagctggagcgtcgcacccagtactaccgcgagaacccagacgctccactgccagaccacgagcagaagccagagctgatcgagttcgttaaccaggcagtttacggcggcctgccagcttcccgcgaggttgaccgcccaccaaagttcaaggaaggcgacgttgttcgcttctccaccgcatccccaaagggtcacgcacgccgcgctcgctacgttcgcggcaagaccggcaccgttgttaagcaccacggcgcatacatctacccagacaccgctggtaacggcctgggcgagtgcccagagcacctgtacaccgttcgcttcaccgagcaggagctgtggggtccagagggcgacccaaactcctccgtttactacgactgctgggagccatacatcgagctggttgacaccaaggcagctgcagcttga

SEQ ID NO: 5 - DNA, 618 bp, synthetic construct
  source 1..618: mol_type = other DNA; note = Variant DSM 43832 nhhA
atgaccgagaacatcacccgcaagtccgacgaggagatccagaaggagatcaccgttcgcgttaaggctctggagtccatgctgatcgagcagggcatcctgaccacctctatgatcgaccgcatggcagagatctacgagaacgaggttggcccacacctgggcgctaaggttgttgttaaggcatggaccgacccagagttcaagaagcgcctgctggctgacggtaccgaggcatgcaaggagctgggtatcggcggcctgcagggcgaggacatgatgtgggttgagaacaccgacgaggttcaccacgttgttgtttgcaccctgtgctcctgctacccatggccagttctgggcctgccaccaaactggtacaaggagccacagtaccgctcccgcgttgttcgcgagccacgccagctgctgaaggaagagttcggcttcgaggttccaccatccaaggagatcaaggtttgggactcctcctccgagatgcgcttcgttgttctgccacagcgcccagctggtaccgacggttggtccgaagaggagctggcaaccctggttacccgcgagtccatgatcggcgttgagccagcaaaggctgttgcctga

SEQ ID NO: 6 - DNA, 435 bp, synthetic construct
  source 1..435: mol_type = other DNA; note = Variant DSM 43832 nhhG activator protein
atgtccgctgaggcaaaggttcgcctgaagcactgcccaaccgcagaggaccgcgcagctgcagacgcactgctggctcagctgccaggcggcgaccgcgcactggaccgcggcttcgacgagccatggcagctgcgcgctttcgcactggctgttgcagcatgccgcgcaggccgcttcgagtggaagcagctgcagcaggctctgatctcctccatcggcgagtgggagcgcacccacgacctggacgacccatcctggtcctactacgagcacttcgttgctgcactggagtccgttctgggcgaggaaggcatcgttgagccagaggcactggacgagcgcaccgcagaggttctggctaacccaccaaacaaggaccaccacggcccacacctggagccagttgcagttcacccagctgttcgctcctaa

SEQ ID NO: 7 - DNA, 1792 bp, synthetic construct
  source 1..1792: mol_type = other DNA; note = Variant DSM 43832 nhhBAG operon
catatgaacggcgtgtacgacgttggtggcaccgatggtctgggtccgattaaccgtccggcggatgagccggtgttccgtgcggagtgggaaaaggttgcgttcgcgatgtttccggcgaccttccgtgcgggctttatgggtctggatgagttccgttttggtattgaacagatgaacccggcggagtacctggaaagcccgtactattggcactggatccgtacctatattcaccacggcgtgcgtaccggcaagatcgacctggaggaactggagcgtcgtacccaatactatcgtgaaaacccggatgcgccgctgccggatcatgaacagaaaccggagctgattgaattcgtgaaccaggcggtttatggtggcctgccggcgagccgtgaggtggaccgtccgccgaagttcaaagaaggcgatgtggttcgttttagcaccgcgagcccgaagggtcatgcgcgtcgtgcgcgttatgttcgtggcaagaccggtaccgtggttaaacaccacggtgcgtacatctatccggacaccgcgggtaacggcctgggcgagtgcccggaacacctgtacaccgttcgttttaccgaacaagaactgtggggtccggagggtgacccgaacagcagcgtgtactatgattgctgggagccgtatattgaactggttgataccaaagcggcggcggcgtgaaaggagatatagatatgaccgaaaacatcacccgtaagagcgacgaggaaatccagaaagagattaccgtgcgtgttaaggcgctggaaagcatgctgatcgagcaaggtattctgaccaccagcatgatcgatcgtatggcggaaatttacgaaaacgaagtgggtccgcacctgggtgcgaaggtggttgtgaaagcgtggaccgacccggagttcaagaaacgtctgctggcggatggcaccgaagcgtgcaaagagctgggtattggtggcctgcagggcgaagacatgatgtgggtggaaaacaccgatgaggttcaccacgttgtggtttgcaccctgtgcagctgctatccgtggccggttctgggtctgccgccgaactggtacaaagaaccgcagtatcgtagccgtgtggttcgtgagccgcgtcaactgctgaaagaggagttcggctttgaagtgccgccgagcaaggagatcaaagtttgggacagcagcagcgagatgcgttttgtggttctgccgcaacgtccggcgggtaccgatggttggagcgaagaggagctggcgaccctggtgacccgtgaaagcatgattggtgtggagccggcgaaggcggttgcgtgaaaggagatatagatatgagcgcggaggcgaaagtgcgtctgaaacactgcccgaccgcggaagatcgtgcggcggcggatgcgctgctggcgcagctgccgggtggcgaccgtgcgctggatcgtggtttcgacgagccgtggcaactgcgtgcgtttgcgctggcggttgcggcgtgccgtgcgggtcgtttcgaatggaagcagctgcagcaagcgctgatcagcagcattggcgagtgggaacgtacccacgatctggacgatccgagctggagctactatgagcactttgtggcggcgctggaaagcgttctgggcgaggaaggcatcgtggagccggaagcgctggatgagcgtaccgcggaagttctggcgaacccgccgaacaaagaccaccacggcccgcacctggagccggtggcggttcacccggcggtgcgtagctaactcgag

SEQ ID NO: 8 - DNA, 1747 bp, synthetic construct
  source 1..1747: mol_type = other DNA; note = Wildtype DSM 43832 nhhBAG operon
atgaacggcgtgtacgacgtcggcggcaccgatgggctgggcccgatcaaccggcccgcggacgaaccggtcttccgcgccgagtgggagaaggtcgcgttcgcgatgttcccggcgacgttccgggccggcttcatgggcctggacgagttccggttcggcatcgagcagatgaacccggccgagtacctcgagtcgccgtactactggcactggatccgcacctacatccaccacggcgtccgcaccggcaagatcgatctcgaggagctggagcgccgcacgcagtactaccgggagaaccccgacgccccgctgcccgagcacgagcagaagccggagttgatcgagttcgtcaaccaggccgtctacggcgggctgcccgcaagccgggaggtcgaccgaccgcccaagttcaaggagggcgacgtggtgcggttctccaccgcgagcccgaagggccacgcccggcgcgcgcggtacgtgcgcggcaagaccgggacggtggtcaagcaccacggcgcgtacatctacccggacaccgccggcaacggcctgggcgagtgccccgagcacctctacaccgtccgcttcacggcccaggagctgtgggggccggaaggggacccgaactccagcgtctactacgactgctgggagccctacatcgagctcgtcgacacgaaggcggccgcggcatgaccgagaacatcctgcgcaagtcggacgaggagatccagaaggagatcacggcgcgggtcaaggccctggagtcgatgctcatcgaacagggcatcctcaccacgtcgatgatcgaccggatggccgagatctacgagaacgaggtcggcccgcacctcggcgcgaaggtcgtcgtgaaggcctggaccgacccggagttcaagaagcgtctgctcgccgacggcaccgaggcctgcaaggagctcggcatcggcggcctgcagggcgaggacatgatgtgggtggagaacaccgacgaggtccaccacgtcgtcgtgtgcacgctctgctcctgctacccgtggccggtgctggggctgccgccgaactggttcaaggagccgcagtaccgctcccgcgtggtgcgtgagccccggcagctgctcaaggaggagttcggcttcgaggtcccgccgagcaaggagatcaaggtctgggactccagctccgagatgcgcttcgtcgtcctcccgcagcgccccgcgggcaccgacgggtggagcgaggaggagctcgccaccctcgtcacccgcgagtcgatgatcggcgtcgaaccggcgaaggcggtcgcgtgagcgccgaggcgaaggtccgcctgaagcactgccccacggccgaggaccgggcggcggccgacgcgctgctcgcgcagctgcccggcggcgaccgcgcgctcgaccgcggcttcgacgagccgtggcagctgcgggcgttcgcgctggcggtcgcggcgtgcagggcgggccggttcgagtggaagcagctgcagcaggcgctgatctcctcgatcggggagtgggagcgcacccacgatctcgacgatccgagctggtcctactacgagcacttcgtcgccgcgctggaatccgtgctcggcgaggaagggatcgtcgagccggaggcgctggacgagcgcaccgcggaggtcttggccaacccgccgaacaaggatcaccatggaccgcatctggagcccgtcgcggtccacccggccgtgcggtcctga

SEQ ID NO: 9 - protein, 233 aa, synthetic construct
  source 1..233: note = Wildtype nhhB
MNGVYDVGGTDGLGPINRPADEPVFRAEWEKVAFAMFPATFRAGFMGLDEFRFGIEQMNPAEYLESPYYWHWIRTYIHHGVRTGKIDLEELERRTQYYRENPDAPLPEHEQKPELIEFVNQAVYGGLPASREVDRPPKFKEGDVVRFSTASPKGHARRARYVRGKTGTVVKHHGAYIYPDTAGNGLGECPEHLYTVRFTAQELWGPEGDPNSSVYYDCWEPYIELVDTKAAAA

SEQ ID NO: 10 - protein, 205 aa, synthetic construct
  source 1..205: note = Wildtype nhhA
MTENILRKSDEEIQKEITARVKALESMLIEQGILTTSMIDRMAEIYENEVGPHLGAKVVVKAWTDPEFKKRLLADGTEACKELGIGGLQGEDMMWVENTDEVHHVVVCTLCSCYPWPVLGLPPNWFKEPQYRSRVVREPRQLLKEEFGFEVPPSKEIKVWDSSSEMRFVVLPQRPAGTDGWSEEELATLVTRESMIGVEPAKAVA

SEQ ID NO: 11 - DNA, 1783 bp, synthetic construct
  source 1..1783: mol_type = other DNA; note = Variant NHase operon
atgaacggcgtttacgacgttggcggcaccgacggcctgggtccaatcaaccgcccagcagacgagccagttttccgcgctgagtgggagaaggttgcattcgctatgttcccagcaaccttccgcgctggcttcatgggcctggacgagttccgcttcggcatcgagcagatgaacccagcagagtacctggagtccccatactactggcactggatccgcacctacatccaccacggcgttcgcaccggcaagatcgacctggaggagctggagcgtcgcacccagtactaccgcgagaacccagacgctccactgccagaccacgagcagaagccagagctgatcgagttcgttaaccaggcagtttacggcggcctgccagcttcccgcgaggttgaccgcccaccaaagttcaaggaaggcgacgttgttcgcttctccaccgcatccccaaagggtcacgcacgccgcgctcgctacgttcgcggcaagaccggcaccgttgttaagcaccacggcgcatacatctacccagacaccgctggtaacggcctgggcgagtgcccagagcacctgtacaccgttcgcttcaccgagcaggagctgtggggtccagagggcgacccaaactcctccgtttactacgactgctgggagccatacatcgagctggttgacaccaaggcagctgcagcttgaaaggagatatagatatgaccgagaacatcacccgcaagtccgacgaggagatccagaaggagatcaccgttcgcgttaaggctctggagtccatgctgatcgagcagggcatcctgaccacctctatgatcgaccgcatggcagagatctacgagaacgaggttggcccacacctgggcgctaaggttgttgttaaggcatggaccgacccagagttcaagaagcgcctgctggctgacggtaccgaggcatgcaaggagctgggtatcggcggcctgcagggcgaggacatgatgtgggttgagaacaccgacgaggttcaccacgttgttgtttgcaccctgtgctcctgctacccatggccagttctgggcctgccaccaaactggtacaaggagccacagtaccgctcccgcgttgttcgcgagccacgccagctgctgaaggaagagttcggcttcgaggttccaccatccaaggagatcaaggtttgggactcctcctccgagatgcgcttcgttgttctgccacagcgcccagctggtaccgacggttggtccgaagaggagctggcaaccctggttacccgcgagtccatgatcggcgttgagccagcaaaggctgttgcctgaaaggagatatagatatgtccgctgaggcaaaggttcgcctgaagcactgcccaaccgcagaggaccgcgcagctgcagacgcactgctggctcagctgccaggcggcgaccgcgcactggaccgcggcttcgacgagccatggcagctgcgcgctttcgcactggctgttgcagcatgccgcgcaggccgcttcgagtggaagcagctgcagcaggctctgatctcctccatcggcgagtgggagcgcacccacgacctggacgacccatcctggtcctactacgagcacttcgttgctgcactggagtccgttctgggcgaggaaggcatcgttgagccagaggcactggacgagcgcaccgcagaggttctggctaacccaccaaacaaggaccaccacggcccacacctggagccagttgcagttcacccagctgttcgctcctaa

(19) United States
(12) Patent Application Publication
(10) Pub. No.: US 2025/0297293 A1
(43) Pub. Date: Sep. 25, 2025

(54) VARIANT NITRILE HYDRATASES, MICROBIA WHICH EXPRESS SAME, AND USE IN AMIDE SYNTHESIS

(71) Applicant: KEMIRA OYJ, HELSINKI (FI)

(72) Inventors: Jaakko SIMELL, Helsinki (FI); Jan MODREGGER, Vienna (AT); Axel NIEBISCH, Vienna (AT)

(21) Appl. No.: 18/848,942

(22) PCT Filed: Mar. 21, 2023
(86) PCT No.: PCT/US2023/064740

§ 371 Date: Sep. 20, 2024

Related U.S. Application Data
(60) Provisional application No. 63/322,203, filed on Mar. 21, 2022.

(51) Int. Cl. C12P 13/02; C12N 9/88

(52) U.S. Cl. CPC C12P 13/02; C12N 9/88; C12Y 402/01084

(57) ABSTRACT

The present invention relates to a variant nitrile hydratase which is engineered to comprise greater activity and/or stability, nucleic acids encoding said nitrile hydratase, and microbia engineered to express said novel nitrile hydratase. Additionally the invention relates to the use of this nitrile hydratase and microbia which express said nitrile hydratase as a biocatalyst, particularly in methods for producing an amide compound from a nitrile compound, preferably for use in converting acrylonitrile to acrylamide.

### CROSS-REFERENCE TO RELATED APPLICATIONS

This application is a U.S. National Phase application of Int'l Appl. No. PCT/US2023/064740, filed Mar. 21, 2023, which claims the benefit to U.S. Provisional Appl. No. 63/322,203, filed Mar. 21, 2022, each of which are incorporated herein by reference in their entireties.

### SEQUENCE LISTING DISCLOSURE

The contents of the electronic sequence listing (11497040018001.xml; Size: 20,613 bytes; and Date of Creation: Sep. 12, 2024) is herein incorporated by reference in its entirety.

### FIELD OF THE INVENTION

The present invention relates to a novel modified nitrile hydratase and/or operon containing which is/are engineered to comprise greater activity and/or stability, nucleic acids encoding said modified nitrile hydratase and/or operon, and microbia engineered to comprise and express said modified nitrile hydratase or modified nitrile hydratase containing operon. Additionally the invention relates to the use of this modified nitrile hydratase and/or microbia which are engineered to express said modified nitrile hydratase as biocatalysts, particularly in methods for producing an amide compound from a nitrile compound, preferably for use in converting acrylonitrile to acrylamide.

### BACKGROUND OF THE INVENTION

Acrylamide is used as a monomer to form polymers and copolymers of acrylamide. For these polymerization and copolymerization reactions aqueous acrylamide solutions prepared by bioconversion can be used. Since the discovery of nitrile hydratase, a microbial enzyme that hydrolyses nitriles to amides, microorganisms having nitrile hydratase activity have been intensively used for the industrial production of amide compounds. Due to milder reaction conditions compared to the chemical synthesis of amides, the use of nitrile hydratase producing microorganisms as biocatalysts is more and more on the rise.

In fact, one of the most well-known commercial examples of nitrile bioconversion by nitrile hydratase producing microorganisms is the manufacture of acrylamide (AMD) from acrylonitrile (AN). However, a challenging problem in the use of nitrile hydratase producing microorganisms as biocatalysts, is that both AN and AMD deactivate the biocatalyst. Accordingly providing nitrile hydratase biocatalysts which remain stable for prolonged duration is problematic. Also, nitrile hydratase biocatalysts which are modified to enhance stability often are not as active as the unmodified biocatalyst, i.e., they do not start the conversion of AN to AMD as rapidly as the unmodified biocatalyst.

Therefore, the technical problem underlying the present invention is to provide improved nitrile hydratases and nitrile hydratase producing microorganisms and their use as biocatalysts, i.e., which biocatalysts when used to covert nitriles into amides possess both enhanced stability (resistance to AN and AMD deactivation) and desirable activity (i.e., start the conversion of AN to AMD very rapidly, i.e., wherein biocatalyst activity is the initial reaction rate).

The technical problem is solved by providing the embodiments reflected in the claims, described in the description and illustrated in the examples and figures that follow.

### SUMMARY OF THE INVENTION

The present invention relates to a novel nitrile hydratase which is engineered to comprise greater activity and/or stability, nucleic acids encoding said nitrile hydratase, and microbia engineered to express said novel nitrile hydratase. Additionally the invention relates to the use of this nitrile hydratase and microbia which are engineered to express said nitrile hydratase as biocatalysts, particularly in methods for producing an amide compound from a nitrile compound, preferably for use in converting acrylonitrile to acrylamide.

More specifically the invention relates to a novel nitrile hydratase derived from Pseudonocardia thermophila which has been engineered to comprise mutations which provide for enhanced stability and activity. In short we have created a novel nitrile hydratase enzyme-coding DNA-sequence which may be transferred to another microorganism, e.g., an industrial production microorganism and used in industrial production of Acrylamide (AMD) from Acrylonitrile (AN).

Even more specifically the invention provides a variant nitrile hydratase (enzyme comprising an alpha subunit (nhhA) having an amino acid sequence which at least 98, 99 or 100% sequence identical to SEQ ID NO: 2 and a beta subunit (nhhB) comprising an amino acid sequence which at least 98, 99 or 100% sequence identical to SEQ ID NO: 1, with the proviso that the alpha subunit comprises one, two or three of the following mutations: L6T, A19V and F126Y and the beta subunit comprises one or both of the following mutations: E108D and A200E or comprises all three of the following mutations E108R, A200E and S212Y; wherein said variant nitrile hydratase possesses enhanced stability and/or activity compared to nitrile hydratase enzyme produced by the wild-type strain (Pseudonocardia thermophila DSM 43832 strain).

In exemplary embodiments the invention provides a variant nitrile hydratase enzyme, wherein the alpha subunit comprises the following mutations: L6T, A19V and F126Y and the beta subunit comprises the following mutations: E108D and A200E; or E108R, A200E and S212Y.

In exemplary embodiments the invention provides a variant nitrile hydratase enzyme, wherein the alpha and beta enzyme subunits are expressed in association with an nhhG activator protein, optionally one comprising an amino acid sequence which at least 98, 99 or 100% sequence identical to SEQ ID NO: 3.

In some exemplary embodiments the invention provides a variant nitrile hydratase according to any the foregoing which comprises a soluble enzyme.

In some exemplary embodiments the variant nitrile hydratase according to any of the foregoing may be immobilized to a solid support.

In some exemplary embodiments the invention provides a variant nitrile hydratase according to any the foregoing which is encapsulated e.g., in a vesicle, sol-gel matrix, or other material that provides for improved thermal stability compared to the enzyme in solution.

In other exemplary embodiments the invention provides nucleic acids comprising sequences encoding an nitrile hydratase comprising an alpha subunit and beta subunit and optionally an activator protein according to any of the foregoing, wherein the nucleic acids encoding one or more of the alpha subunit, beta subunit and optionally the activator protein are codon optimized to increase expression in a desired microorganism, optionally a yeast, fungus or bacterium.

In other exemplary embodiments the invention provides nucleic acids as above which are codon optimized for expression in a desired microorganism optionally a bacterium selected from Rhodococcus, Aspergillus, Acidovorax, Agrobacterium, Bacillus, Bradyrhizobium, Brevibacterium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus. In exemplary embodiments of the invention, the microorganism may be selected from bacteria of the genus Rhodococcus, Pseudomonas, Escherichia and Geobacillus or optionally is selected from the following species Rhodococcus rhodochrous, Rhodococcus pyridinovorans, Rhodococcus erythropolis, Rhodococcus equi, Rhodococcus ruber, Rhodococcus opacus, Aspergillus niger, Acidovorax avenae, Acidovorax facilis, Agrobacterium tumefaciens, Agrobacterium radiobacter, Bacillus subtilis, Bacillus pallidus, Bacillus smithii, Bacillus sp BR449, Bradyrhizobium oligotrophicum, Bradyrhizobium diazoefficiens, Bradyrhizobium japonicum, Burkholderia cenocepacia, Burkholderia gladioli, Escherichia coli, Geobacillus sp. RAPc8, Klebsiella oxytoca, Klebsiella pneumonia, Klebsiella variicola, Mesorhizobium ciceri, Mesorhizobium opportunistum, Mesorhizobium sp F28, Moraxella, Pantoea endophytica, Pantoea agglomerans, Pseudomonas chlororaphis, Pseudomonas putid, Rhizobium, Rhodopseudomonas palustris, Serratia liquefaciens, Serratia marcescens, Amycolatopsis, Arthrobacter, Brevibacterium sp CH1, Brevibacterium sp CH2, Brevibacterium sp R312, Brevibacterium imperiale, Corynebacterium nitrilophilus, Corynebacterium pseudodiphteriticum, Corynebacterium glutamicum, Corynebacterium hoffmanii, Microbacterium imperiale, Microbacterium smegmatis, Micrococcus luteus, Nocardia globerula, Nocardia rhodochrous, Pseudonocardia thermophila, Trichoderma, Myrothecium verrucaria, Aureobasidium pullulans, Candida famata, Candida guilliermondii, Candida tropicalis, Cryptococcus flavus, Cryptococcus sp UFMG-Y28, Debaryomyces hanseii, Geotrichum candidum, Geotrichum sp JR1, Hanseniaspora, Kluyveromyces thermotolerans, Pichia kluyveri, Rhodotorula glutinis, Comomonas testosterone, Pyrococcus abyssi, Pyrococcusfuriosus, Pyrococcus horikoshii, Brevibacterium casei, or Nocardia sp. 163; and in specific exemplary embodiments may comprise a bacterium of the species Corynebacterium glutamicum, further optionally strain C. glutamicum ATCC13032 or its derivative MB001(DE3).

In other exemplary embodiments the invention provides nucleic acids as above, wherein:
i) the beta subunit is encoded by the nucleic acid of SEQ ID NO: 4;
ii) the alpha subunit is encoded by the nucleic acid of SEQ ID NO: 5;
iii) the activator protein is encoded by the nucleic acid of SEQ ID NO:6; or
iv) any combination of (i) to (iii).

In other exemplary embodiments the invention provides an nitrile hydratase operon comprising nucleic acids which encode for the alpha (nhhA) and beta (nhhB) subunits of a variant nitrile hydratase and optionally an activator protein (nhhG) according to any of the foregoing, optionally wherein said nucleic acids are those of any one of the foregoing, optionally which operon is derived from a yeast, fungus or bacterium that expresses nitrile hydratase, optionally a Pseudonocardia bacterium, further optionally Pseudonocardia thermophila.

In other exemplary embodiments the invention provides an nitrile hydratase operon comprising nucleic acids which encode for the alpha (nhhA) and beta (nhhB) subunits of a variant nitrile hydratase and optionally an activator protein (nhhG) according to any of the foregoing, which comprises SEQ ID NO: 7.

In other exemplary embodiments the invention provides one or more extrachromosomal sequences, optionally plasmids, comprising at least one nucleic acid or operon according to any of those previously described above.

In other exemplary embodiments the invention provides a microorganism, optionally a yeast, fungus or bacterium, further optionally an industrial microorganism which optionally does not endogenously express nitrile hydratase or endogenously expresses nitrile hydratase, which microorganism is engineered to comprise nucleic acids encoding a nitrile hydratase comprising alpha (nhhA) and beta (nhhB) subunits of a variant nitrile hydratase and optionally an activator protein (nhhG) according to any of the foregoing or an operon comprising said nucleic acids, optionally as above described, optionally wherein one or more of said nucleic acids are comprised in one or more extrachromosomal sequences (plasmids) or are integrated (one or more copies) into the chromosomal DNA of the microorganism.

In some exemplary embodiments the microorganism is an industrial microorganism which endogenously expresses nitrile hydratase, and said nucleic acids or operon replaces the endogenous nitrile hydratase gene or operon comprising the endogenous nitrile hydratase gene. Microorganisms encoding nitrile hydratase include microbial species by way of example Rhodococcus, Aspergillus, Acidovorax, Agrobacterium, Bacillus, Bradyrhizobium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus.

In some exemplary embodiments the microorganism used to express the variant nitrile hydratase or nitrile hydratase operon is a microbe selected from Rhodococcus, Pseudomonas, Escherichia, and Geobacillus. Typically, the biocatalyst is selected from the group consisting of Rhodococcus, Aspergillus, Acidovorax, Agrobacterium, Bacillus, Bradyrhizobium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus, or any part of said microorganism having nitrile hydratase activity or the microorganism used to express the variant nitrile hydratase or nitrile hydratase operon is a bacterium selected from Rhodococcus, Aspergillus, Acidovorax, Agrobacterium, Bacillus, Bradyrhizobium, Brevibacterium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus.

In more specific exemplary embodiments of the invention, the microorganism may be selected from bacteria of the genus Rhodococcus, Pseudomonas, Escherichia and Geobacillus or is selected from the following species Rhodococcus rhodochrous, Rhodococcus pyridinovorans, Rhodococcus erythropolis, Rhodococcus equi, Rhodococcus ruber, Rhodococcus opacus, Aspergillus niger, Acidovorax avenae, Acidovorax facilis, Agrobacterium tumefaciens, Agrobacterium radiobacter, Bacillus subtilis, Bacillus pallidus, Bacillus smithii, Bacillus sp BR449, Bradyrhizobium oligotrophicum, Bradyrhizobium diazoefficiens, Bradyrhizobium japonicum, Burkholderia cenocepacia, Burkholderia gladioli, Escherichia coli, Geobacillus sp. RAPc8, Klebsiella oxytoca, Klebsiella pneumonia, Klebsiella variicola, Mesorhizobium ciceri, Mesorhizobium opportunistum, Mesorhizobium sp F28, Moraxella, Pantoea endophytica, Pantoea agglomerans, Pseudomonas chlororaphis, Pseudomonas putid, Rhizobium, Rhodopseudomonas palustris, Serratia liquefaciens, Serratia marcescens, Amycolatopsis, Arthrobacter, Brevibacterium sp CH1, Brevibacterium sp CH2, Brevibacterium sp R312, Brevibacterium imperiale, Corynebacterium nitrilophilus, Corynebacterium pseudodiphteriticum, Corynebacterium glutamicum, Corynebacterium hoffmanii, Microbacterium imperiale, Microbacterium smegmatis, Micrococcus luteus, Nocardia globerula, Nocardia rhodochrous, Pseudonocardia thermophila, Trichoderma, Myrothecium verrucaria, Aureobasidium pullulans, Candida famata, Candida guilliermondii, Candida tropicalis, Cryptococcus flavus, Cryptococcus sp UFMG-Y28, Debaryomyces hanseii, Geotrichum candidum, Geotrichum sp JR1, Hanseniaspora, Kluyveromyces thermotolerans, Pichia kluyveri, Rhodotorula glutinis, Comomonas testosterone, Pyrococcus abyssi, Pyrococcusfuriosus, Pyrococcus horikoshii, Brevibacterium casei, or Nocardia sp. 163.

In an even more specific exemplary embodiment the biocatalyst is a bacterium of the species Corynebacterium glutamicum, optionally strain C. glutamicum ATCC13032 or its derivative MB001(DE3) which has been deposited in the German Collection of Microorganisms and Cell Cultures (DSMZ) under strain No. 102071.

In some exemplary embodiments the invention provides methods for producing an amide compound from a nitrile compound, the method comprising: contacting the nitrile compound with an nitrile hydratase or a bacterium which expresses said nitrile hydratase according to any of the above described. The bacterium optionally may be in dried form. The enzyme may be in soluble form or immobilized such as to a solid support. Further optionally the nitrile hydratase may be encapsulated with a material such as a gel-sol matrix that enhances thermostability during amide synthesis.

In some exemplary embodiments the invention provides methods for producing an amide compound from a nitrile compound, comprising: contacting the nitrile compound with an nitrile hydratase or a bacterium which expresses said nitrile hydratase according to any of the above described wherein the amide compound is selected from the group consisting of acrylamide, methacrylamide, acetamide, and nicotinamide and preferably is acrylamide, and the nitrile compound is selected from the group consisting of acrylonitrile, methacrylonitrile, acetonitrile, and 3-cyanopyridine and preferably is acrylonitrile.

In some exemplary embodiments these amide synthesis methods use a soluble nitrile hydratase, an encapsulated nitrile hydratase, an immobilized nitrile hydratase, or uses a whole microbial cell biocatalyst or a lysed microbial cell biocatalyst.

In some exemplary embodiments these amide synthesis methods use an intact microbe biocatalyst which optionally may be fresh (i.e., straight from fermentation); stored, e.g., stored as frozen (frozen as wet); or dry such as a lyophilizate.

Exemplary methods for producing an amide compound from a nitrile compound using a biocatalyst according to the invention are further described and exemplified infra.

### BRIEF DESCRIPTION OF THE DRAWINGS

The invention will be described in more detail with reference to appended drawings, described in detail below.

FIG. 1 contains an alignment of wild-type (DSM 43832 strain) and variant nitrile hydratase Beta Subunit (nhhB) polypeptide sequences.

FIG. 2 contains an alignment of wild-type (DSM 43832 strain) and variant nitrile hydratase Alpha Subunit (nhhA) polypeptide sequences.

FIG. 3 contains an alignment of wild-type (DSM 43832 strain) nhhBAG operon and variant nhhBAG operon nucleic acid sequences. The variant nhhBAG operon includes NdeI/XhoI restriction sites used for cloning DSM 43832 Strain nhhBAG operon and lacks the amidase gene present in endogenous operon upstream of nhhB. In the wildtype operon sequence the TGA stop codon of nhhB overlaps with the ATG start codon of nhhA and similarly the TGA stop codon of nhhA overlaps with the GTG start codon of nhhG. By contrast in the variant operon, this overlap was resolved by introduction of intergenic ribosomal binding sites (seen as gaps in the alignment.

### DETAILED DESCRIPTION OF THE INVENTION

Before describing the invention, the following definitions are provided. Unless stated otherwise all terms are to be construed as they would be by a person skilled in the art.

### Definitions

As used herein, the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

As used herein the term “biocatalyst” refers to any biocatalyst having nitrile hydratase activity. The biocatalyst capable of converting acrylonitrile to acrylamide may be a microorganism which encodes an enzyme having nitrile hydratase activity or any part of said microorganism having nitrile hydratase activity. The biocatalyst may be selected from said microorganism, lysed cells of said microorganism, a cell lysate of said microorganism, or any combination of these. In a very specific embodiment, the biocatalyst is a variant nitrile hydratase as disclosed in the examples or a bacterial strain (Corynebacterium glutamicum) expressing same.

As used herein the term “biomass” generally refers to collected cells, generally microbial cells and most typically bacterial cells, obtained after a fermentation, typically after excess broth has been removed, wherein said removal is optionally effected by filtration or centrifugation, typically resulting in a biomass composition having a dry content ranging from about 10-35%, more typically around 25-30%.

As used herein, the term “microorganism(s)”, when used herein encompasses “nitrile hydratase producing microorganism(s)”, wherein said microorganisms endogenously express and/or are engineered to express a variant nitrile hydratase according to the invention. Such microorganism in the context of the present invention is preferably a bacterium, fungus or yeast. Within the present invention “nitrile hydratase producing microorganisms” are used, or are for use, as a biocatalyst for converting a nitrile compound into the corresponding amide compound.

As used herein, the term “nitrile compound” is one converted by a nitrile hydratase according to the invention or a microorganism which expresses a nitrile hydratase according to the present invention into an amide compound by the action of said nitrile hydratase. A nitrile compound is any organic compound that has a —C═N functional group such as methacrylonitrile, acetonitrile or 3-cyanopyridine and preferably acrylonitrile.

As used herein, the term “amide compound” is a compound produced by nitrile hydratase from a nitrile compound. An amide compound has the functional group RₙC(O)ₓNR′₂, wherein R and R′ refer to H or organic groups, or organic amides and n=1, x=1. Examples of such amide compounds include methacrylamide, acetamide or nicotinamide and preferably comprises acrylamide.

As used herein, the term “nitrile hydratase producing microorganism” may be any microorganism which is able to produce the inventive variant nitrile hydratase. In the context of the present invention, “nitrile hydratase producing microorganisms” include those not naturally encoding nitrile hydratase which are genetically engineered to contain a gene or polynucleotide encoding a nitrile hydratase (e.g., via transformation, transduction, transfection, conjugation, or other methods suitable to transfer or insert a polynucleotide into a cell as known in the art; cf. Sambrook and Russell 2001, Molecular Cloning: A Laboratory Manual, CSH Press, Cold Spring Harbor, N.Y., USA), thus enabling the microorganisms to produce and stably maintain the nitrile hydratase enzyme. For this purpose, it may further be required to insert additional polynucleotides which may be necessary to allow transcription and translation of the nitrile hydratase gene or mRNA, respectively. Such additional polynucleotides may comprise, inter alia, promoter sequences, or replication origins or other plasmid-control sequences. In this context, such genetically engineered microorganisms which naturally do not contain a gene encoding a nitrile hydratase but which have been manipulated such as to contain a polynucleotide encoding a nitrile hydratase may be prokaryotic or eukaryotic microorganisms. Examples of such prokaryotic microorganisms include, e.g., Escherichia coli and Corynebacterium species. Examples for such eukaryotic microorganisms include, e.g., yeast (e.g., Saccharomyces cerevisiae or Pichia pastoris).

“Nitrile hydratase producing microorganisms” which (naturally or non-naturally) encode nitrile hydratase are in some embodiments capable of producing and stably maintaining nitrile hydratase. However, in accordance with the present invention, it is also possible that such microorganisms only produce nitrile hydratase during cultivation (or fermentation) of the microorganisms. Such microbia include, inter alia, bacteria of the genus Rhodococcus, Aspergillus, Acidovorax, Agrobacterium, Bacillus, Bradyrhizobium, Brevibacterium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus. In exemplary embodiments of the invention, the microorganism may be selected from bacteria of the genus Rhodococcus, Pseudomonas, Escherichia and Geobacillus. Also, “nitrile hydratase producing microorganism” include, inter alia, the following species Rhodococcus rhodochrous, Rhodococcus pyridinovorans, Rhodococcus erythropolis, Rhodococcus equi, Rhodococcus ruber, Rhodococcus opacus, Aspergillus niger, Acidovorax avenae, Acidovorax facilis, Agrobacterium tumefaciens, Agrobacterium radiobacter, Bacillus subtilis, Bacillus pallidus, Bacillus smithii, Bacillus sp BR449, Bradyrhizobium oligotrophicum, Bradyrhizobium diazoefficiens, Bradyrhizobium japonicum, Burkholderia cenocepacia, Burkholderia gladioli, Escherichia coli, Geobacillus sp. RAPc8, Klebsiella oxytoca, Klebsiella pneumonia, Klebsiella variicola, Mesorhizobium ciceri, Mesorhizobium opportunistum, Mesorhizobium sp F28, Moraxella, Pantoea endophytica, Pantoea agglomerans, Pseudomonas chlororaphis, Pseudomonas putid, Rhizobium, Rhodopseudomonas palustris, Serratia liquefaciens, Serratia marcescens, Amycolatopsis, Arthrobacter, Brevibacterium sp CH1, Brevibacterium sp CH2, Brevibacterium sp R312, Brevibacterium imperiale, Corynebacterium nitrilophilus, Corynebacterium pseudodiphteriticum, Corynebacterium glutamicum, Corynebacterium hoffmanii, Microbacterium imperiale, Microbacterium smegmatis, Micrococcus luteus, Nocardia globerula, Nocardia rhodochrous, Pseudonocardia thermophila, Trichoderma, Myrothecium verrucaria, Aureobasidium pullulans, Candida famata, Candida guilliermondii, Candida tropicalis, Cryptococcus flavus, Cryptococcus sp UFMG-Y28, Debaryomyces hanseii, Geotrichum candidum, Geotrichum sp JR1, Hanseniaspora, Kluyveromyces thermotolerans, Pichia kluyveri, Rhodotorula glutinis, Comomonas testosterone, Pyrococcus abyssi, Pyrococcusfuriosus, Pyrococcus horikoshii, Brevibacterium casei, or Nocardia sp. 163.

In specific exemplary embodiments the “nitrile hydratase producing microorganism” is a bacterium of the species Corynebacterium glutamicum, preferably strain Corynebacterium strain C. glutamicum ATCC13032 or its derivative MB001(DE3) which has been deposited in the German Collection of Microorganisms and Cell Cultures (DSMZ) under strain No. 102071.

In the context of the present invention, “nitrile hydratase” (“Nitrile Hydratase”) refers to a microbial enzyme that catalyzes the hydration of nitriles to their corresponding amides (IUBMB Enzyme Nomenclature EC 4.2.1.84. The terms “nitrile hydratase” and “nitrile hydratase” as used herein also encompass modified or enhanced enzymes which are, e.g., capable of converting a nitrile compound (e.g. acrylonitrile) to an amide compound (e.g. acrylamide) more quickly, or which can be produced at a higher yield/time-ratio, or which are more stable, as long as they are capable to catalyze conversion (i.e. hydration) of a nitrile compound (e.g. acrylonitrile) to an amide compound (e.g. acrylamide). This enzyme generally comprises an alpha subunit (nhhA) and beta subunit (nhhB) which subunits are optionally expressed in association with an activator protein (nhhG). In exemplary embodiments it refers to a variant nitrile hydratase (enzyme comprising an alpha subunit (nhhA) having an amino acid sequence which possesses at least 98, 99 or 100% sequence identity to SEQ ID NO: 2 and a beta subunit (nhhB) comprising an amino acid which possesses at least 98, 99 or 100% sequence identity to SEQ ID NO: 1, with the proviso that the alpha subunit comprises one, two or three of the following mutations: L6T, A19V and F126Y and the beta subunit comprises one or both of the following mutations: E108D and A200E or comprises all three of the following mutations E108R, A200E and S212Y; wherein said variant nitrile hydratase possesses enhanced stability and/or activity compared to nitrile hydratase enzyme produced by the wild-type strain (Pseudonocardia thermophila DSM 43832 strain).

In the context of the present invention, “nitrile hydratase stability” or “enzyme stability” or “stability” refer to how well the biocatalyst tolerates AN and AMD under specific reaction conditions (e.g., temperature, solvent etc.), i.e., a good stability means under specific reaction conditions means that the nitrile hydratase has a lower deactivation rate compared to another nitrile hydratase under the same under specific reaction conditions (since both AN and AMD are known to deactivate endogenous nitrile hydratase biocatalysts).

In the context of the present invention, “nitrile hydratase activity” or “enzyme activity” or “activity” refer to the time it takes for the enzyme biocatalyst to start the conversion of AN to AMD. That is to say if the conversion starts very rapidly, the enzyme is said to have high activity, i.e., biocatalyst activity refers to the initial reaction rate.

### DESCRIPTION OF THE INVENTION

### Development of Novel Biocatalyst

We have created a novel nitrile hydratase enzyme and enzyme-coding DNA-sequence and transferred this to an industrial production microorganism. This biocatalyst may be used in industrial production of acrylamide (AMD) from acrylonitrile (AN).

Technically the present inventors sought to obtain a novel nitrile hydratase biocatalyst possessing high activity and which could be used for the production of high concentration, i.e. 54% AMD. As noted previously, while many natural nitrile hydratase enzymes have been sequenced and used for AMD synthesis, typically endogenous nitrile hydratases lack either one or both of the required characteristics (activity and stability).

The development of the inventive biocatalyst included screening of multiple nitrile hydratases and host cells. Host cells, or industrial production microorganisms, are the microbe species where the nitrile hydratase gene is desirably inserted and used in biocatalyst production via fermentation. The development of the novel variant nitrile hydratase and operon included introducing genetic modifications into numerous nitrile hydratase genes and evaluation of the performance (stability/activity) of the variants. Nitrile hydratases originating from several different microbe species were studied. Methods used by the inventors for the improvement of the biocatalyst included in vivo recombination, site specific and random mutagenesis, chaperone co-expression, operon optimization, expression plasmid optimization, codon usage optimization and fermentation optimization.

As is disclosed in the examples, the amino acid sequences of both the alpha and beta nitrile hydratase subunits of an nitrile hydratase endogenously produced by a Pseudonocardia thermophila strain were mutated with the objective of obtaining a variant nitrile hydratase possessing enhanced stability and/or activity compared to the parental nitrile hydratase endogenously produced by the Pseudonocardia thermophila strain.

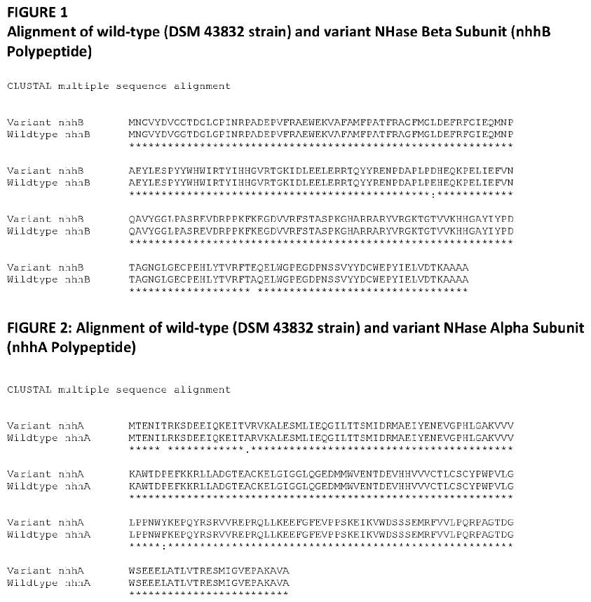

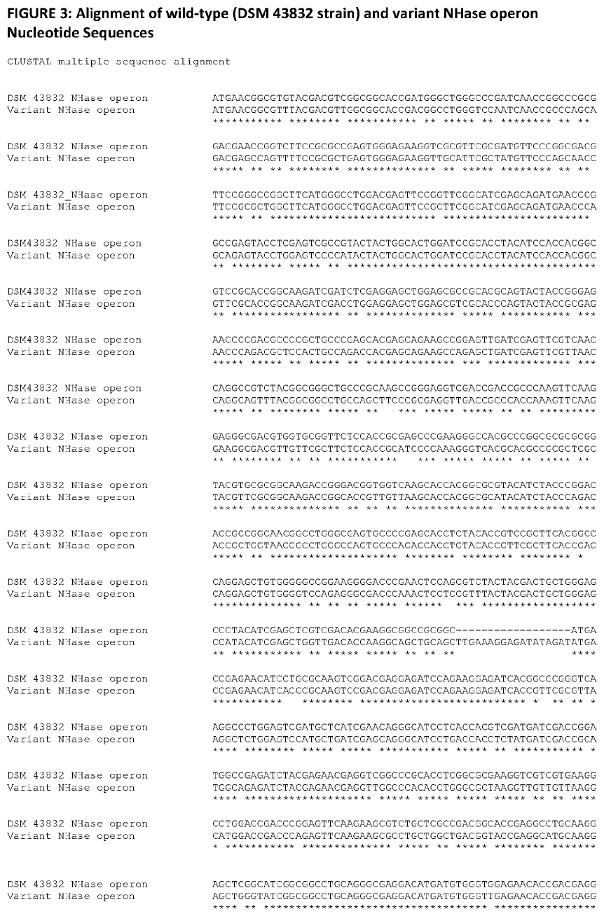

The sequences of the variant nitrile hydratase gene and operon, both the nitrile hydratase gene and the operon comprising were derived from a specific Pseudonocardia thermophila strain and were extensively modified in relation to the wild-type nitrile hydratase genes and the operon containing endogenously comprised in Pseudonocardia thermophila. The alpha and beta nitrile hydratase subunits comprising the specific mutations contained in SEQ ID NO: 2 and SEQ ID NO: 1 (which combinations were selected after screening numerous different combinations of mutations) were found to yield the best combination of enhanced stability and/or activity compared to the parental nitrile hydratase endogenously produced by the Pseudonocardia thermophila strain. (See also the sequence alignment of the wild-type and variant alpha and beta nitrile hydratase subunits contained in FIGS. 1 and 2).

As shown in the examples a variant nitrile hydratase comprising the specific mutations in the alpha subunit contained in SEQ ID NO: 2 (L6T, A19V, F126Y) and the specific mutations in the beta subunit contained in SEQ ID NO: 1 (E108D, A200E) exhibited the best stability/activity and a variant nitrile hydratase comprising the same mutations (L6T, A19V, F126Y) in the alpha subunit contained in SEQ ID NO: 2 and E108R/A200E/S212Y mutations in the beta subunit exhibited the second best stability/activity when compared in amide synthesis experiments disclosed in the examples.

As further described infra in the examples, the nitrile hydratase operon from the Pseudonocardia thermophila strain was further engineered to enhance the stability and/or activity of the nitrile hydratase enzyme and to facilitate expression in a selected exemplary industrial strain, e.g., Corynebacterium glutamicum, optionally strain C. glutamicum ATCC13032 or its derivative MB001(DE3). This parental C. glutamicum MB001(DE3) strain has been deposited in the German Collection of Microorganisms and Cell Cultures (DSMZ) under strain No. 102071. A detailed description of this strain is published in Kortmann M. et al. (2015), “A chromosomally encoded T7 RNA polymerase-dependent gene expression system for Corynebacterium glutamicum: construction and comparative evaluation at the single-cell level”, Microb Biotechnol. 8:253-65.

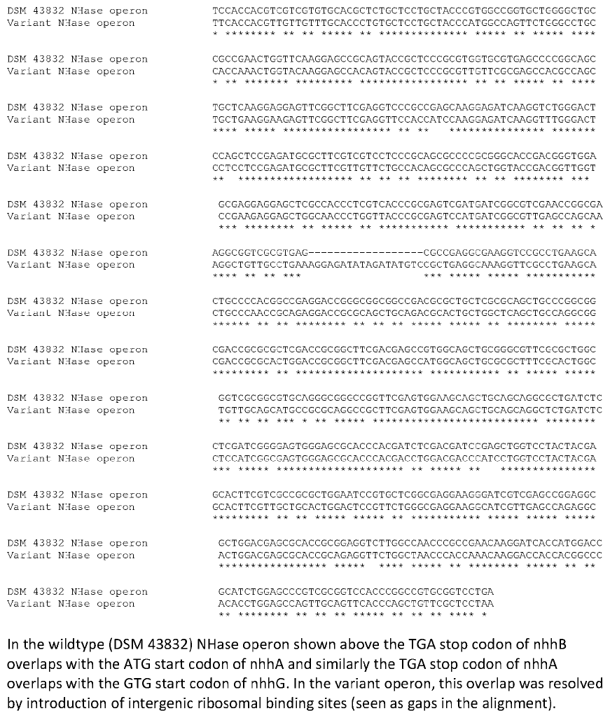

These operon modifications included codon optimization of the parental Pseudonocardia thermophila strain nhhA, nhhB and nhhG coding sequences, eliminating the overlap of the TGA stop codon of nhhB with the ATG start codon of nhhA and the overlap of the TGA stop codon of nhhA with the GTG start codon of nhhG in the variant operon by the introduction of intergenic ribosomal binding sites, introduction of NdeI/XhoI restriction sites used for cloning, deletion of the amidase gene which is part of this operon upstream of nhhB, among other changes. These changes can further be seen from FIG. 3 which contains an alignment of the modified operon and the parental operon of the Pseudonocardia thermophila strain used to derive the modified operon.

Relating to the foregoing, in exemplary embodiments as described in the examples infra, plasmids comprising these variant nitrile hydratase sequences have been transferred to the host strain Corynebacterium glutamicum. As further shown in the examples, exemplary Corynebacterium glutamicum strains which express the variant nitrile hydratase sequences of the present invention when used to produce acrylamide from acrylonitrile were shown to provide for enhanced expression and stability in relation to the parental strain as well as other comparators (e.g., other microbial strains engineered to comprise nitrile hydratases comprising different mutations).

While the exemplary Corynebacterium glutamicum strain which expressed the exemplary variant nitrile hydratase provided for the best activity/stability combination, it is anticipated that the inventive variant nitrile hydratase sequences may be expressed in other microbia, preferably other industrial microbes which are favored for use in industrial processes. Examples thereof a bacterium selected from Rhodococcus, Aspergillus, Acidovorax, Agrobacterium, Bacillus, Bradyrhizobium, Brevibacterium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus or more specifically a microorganism selected from bacteria of the genus Rhodococcus, Pseudomonas, Escherichia and Geobacillus or a bacterium selected from the following species Rhodococcus rhodochrous, Rhodococcus pyridinovorans, Rhodococcus erythropolis, Rhodococcus equi, Rhodococcus ruber, Rhodococcus opacus, Aspergillus niger, Acidovorax avenae, Acidovorax facilis, Agrobacterium tumefaciens, Agrobacterium radiobacter, Bacillus subtilis, Bacillus pallidus, Bacillus smithii, Bacillus sp BR449, Bradyrhizobium oligotrophicum, Bradyrhizobium diazoefficiens, Bradyrhizobium japonicum, Burkholderia cenocepacia, Burkholderia gladioli, Escherichia coli, Geobacillus sp. RAPc8, Klebsiella oxytoca, Klebsiella pneumonia, Klebsiella variicola, Mesorhizobium ciceri, Mesorhizobium opportunistum, Mesorhizobium sp F28, Moraxella, Pantoea endophytica, Pantoea agglomerans, Pseudomonas chlororaphis, Pseudomonas putid, Rhizobium, Rhodopseudomonas palustris, Serratia liquefaciens, Serratia marcescens, Amycolatopsis, Arthrobacter, Brevibacterium sp CH1, Brevibacterium sp CH2, Brevibacterium sp R312, Brevibacterium imperiale, Corynebacterium nitrilophilus, Corynebacterium pseudodiphteriticum, Corynebacterium glutamicum, Corynebacterium hoffmanii, Microbacterium imperiale, Microbacterium smegmatis, Micrococcus luteus, Nocardia globerula, Nocardia rhodochrous, Pseudonocardia thermophila, Trichoderma, Myrothecium verrucaria, Aureobasidium pullulans, Candida famata, Candida guilliermondii, Candida tropicalis, Cryptococcus flavus, Cryptococcus sp UFMG-Y28, Debaryomyces hanseii, Geotrichum candidum, Geotrichum sp JR1, Hanseniaspora, Kluyveromyces thermotolerans, Pichia kluyveri, Rhodotorula glutinis, Comomonas testosterone, Pyrococcus abyssi, Pyrococcus furiosus, Pyrococcus horikoshii, Brevibacterium casei, or Nocardia sp. 163; and optionally comprises a bacterium of the species Corynebacterium glutamicum, further optionally strain C. glutamicum ATCC13032 or its derivative MB001(DE3) which has been deposited in the German Collection of Microorganisms and Cell Cultures (DSMZ) under strain No. 102071.

Novel Biocatalyst Forms which May be Used in Amide Synthesis

The inventive biocatalyst, e.g., exemplary Corynebacterium glutamicum strain which expresses the exemplary variant nitrile hydratase may be fresh (i.e., straight from fermentation); stored, such as stored as frozen (frozen as wet); or dry, e.g., in spray-dried form. In exemplary embodiments during use of the inventive microbial biocatalysts as a biocatalyst we make a slurry by mixing the microbial biocatalyst with water.

After the fermentation, the collected biomass or collected cells may optionally be washed, or otherwise treated e.g. by freezing or drying.

Also, the biocatalyst capable of converting acrylonitrile to acrylamide may be a microorganism which encodes the variant nitrile hydratase or any part of said microorganism having nitrile hydratase activity. The biocatalyst may be selected from said microorganism, lysed cells of said microorganism, a cell lysate of said microorganism, or any combination of these. In a very specific embodiment, the biocatalyst is a nitrile hydratase.

In one embodiment, the amount of the biocatalyst is 0.1 kg dry cells/m3 to 5 kg dry cells/m3 of reaction mixture.

In another embodiment, the amount of the biocatalyst is from 0.1 g dry cells/kg 100% AMD to 3 g dry cells/kg 100% AMD, based on the final AMD amount, more specifically from 0.2 g dry cells/kg 100% AMD to 3 g dry cells/kg 100% AMD, more specifically 0.2 g dry cells/kg 100% AMD to 2.5 g dry cells/kg 100% AMD. In another embodiment the amount of the biocatalyst is from 0.5 g dry cells/kg 100% AMD to 2 g dry cells/kg 100% AMD or more specifically 1.1 g dry cells/kg 100% AMD to 1.5 g dry cells/kg 100% AMD.

Yet in another embodiment the amount of the biocatalyst is from 0.5 g dry cells/kg 50% AMD to 1 g dry cells/kg 50% AMD, based on the final AMD amount, more specifically from 1.6 g dry cells/kg 50% AMD to 1.8 g dry cells/kg 50% AMD.

Yet in another embodiment the amount of the biocatalyst is 0.1 kg dry cells/m3 to 1.5 kg dry cells/m3 of reaction mixture at the end of the maturation of the reaction mixture. In another embodiment the amount of the biocatalyst is 0.1 kg dry cells/m3 to 1.0 kg dry cells/m3 of reaction mixture.

During the process, more of the biocatalyst may be added, for example, if acrylonitrile starts to accumulate in the reactor. The biocatalyst may be added, for example, as a homogenous slurry in water.

### Exemplary Amide Reaction Conditions

The reaction is typically conducted at ambient pressure, more specifically at 1 bar.

A slurry, i.e., an aqueous mixture comprising the biocatalyst, may be produced by any known method in the art, such as mixing water and the biocatalyst in a receptacle or in the reactor. Optionally the slurry is homogenous and not strongly agglomerated.

A reaction of acrylonitrile to acrylamide in aqueous solution in the presence of biocatalyst having nitrile hydratase activity begins once acrylonitrile is fed into a reactor comprising said slurry. In an exemplary embodiment the acrylonitrile may be fed into a reactor comprising said slurry provides a reaction mixture comprising water, acrylamide, acrylonitrile, and biocatalyst. In exemplary embodiments the biocatalyst slurry may be added into the reactor with additional water, and the AN feed commenced after such addition. The amount of additional water and biocatalyst slurry in exemplary embodiments may be about 5000 kg vs. the 100 kg slurry and may range from about 25000 kg vs. the 100 kg slurry to about 1000 kg vs. the 100 kg slurry.

An aqueous solution of acrylamide in high concentration (e.g. at least 35 wt %, or at least 40 wt %, or at least 45 wt %, or at least 50 wt %), at least 55 wt %, at least 56% or at least about 57% or about 55-60% AMD or can be produced with controlled acrylonitrile feed and process temperature profiles. With respect to the foregoing, the maximum AMD concentration can be constrained by precipitation concerns, i.e., at high AMD concentrations it can start to precipitate.

Cooling of the reactor is typically needed to keep the reaction mixture at a desired reaction temperature. The temperature and the acrylonitrile feed rate are each relatively high at the beginning of the reaction to achieve fast reaction rate and short synthesis time. The reactor is started to cool down after, for example, 60 minutes from the start of the reaction since deactivation of the biocatalyst from accumulation of acrylamide is notably lesser in lower temperature compared to higher temperature reaction mixture. The acrylonitrile feed rate is relatively low during the last hours to avoid acrylonitrile accumulation in the reactor.

Because the reaction is exothermic cooling capacity and also safety concerns can be a limiting factor dictating the AN-feed rate. In some exemplary embodiments the amount of AN in the reactor is kept at a maximum of about 3%.

In some exemplary embodiments the reactor containing the biocatalyst is at about 15° C. prior to addition of the AN-feed, the AN-feed is started and the temperature is maintained at about 23° C. until reaction end in order to minimize deactivation of the biocatalyst.

The feeding of acrylonitrile may be continued throughout the process, more specifically continued throughout the process until the maturation phase. Feed rate of the acrylonitrile may vary during the process. The feeding of acrylonitrile may be continuous or intermittent. The feed rate of acrylonitrile depends on the reaction rate of the acrylonitrile to acrylamide and the rate of biocatalyst deactivation. In one embodiment, feeding of the acrylonitrile is continued throughout the process until the maturation phase.

In one embodiment the acrylonitrile feed rate is adjusted during the process to avoid acrylonitrile accumulation into the reaction mixture. The acrylonitrile is fed during the process with such a rate at which the acrylonitrile converts to acrylamide. More specifically the acrylonitrile amount in the reaction mixture is maintained as less than 3 wt %, or less than 2 wt %, more specifically less than 1 wt %, even more specifically less than 0.5 wt % relative to the total amount of reaction mixture. However, in some embodiments the reaction can be conducted at much higher AN-concentrations, e.g., 5%, 10% or even higher if all of the AN is added at once.

In another embodiment of the process, 38% to 48% of total amount of acrylonitrile fed to the reactor is fed during 0 min to 60 min from the beginning of the process; 22% to 30% of total amount of acrylonitrile is fed during 60 min to 120 min of the process; 12% to 18% of total amount of acrylonitrile is fed during 120 min to 180 min of the process; and 8% to 12% of total amount of acrylonitrile is fed during 180 min to 240 min of the process. To reach balance of 100% of fed acrylonitrile, the rest acrylonitrile is fed during the process prior to the maturation phase.

During the maturation phase, substantially no acrylonitrile, and more specifically no acrylonitrile, is fed to the reactor. During maturation, acrylonitrile monomers still present in the reaction mixture react to acrylamide. The reaction mixture is maturated until desired features are reached.

Generally the temperature of the reaction mixture is monitored. The monitoring and measuring may be performed with any suitable means and methods in the art.

In exemplary embodiments the temperature of the reaction mixture is maintained at 15 to 25° C. In one embodiment the temperature is maintained at 19 to 25° C., more specifically at 20 to 22° C. and even more specifically at 22° C. In one embodiment, the temperature is maintained in the desired range by measuring the temperature of the reaction mixture and either cooling the mixture or heating the mixture so that the temperature stays in the desired range. The cooling and/or heating of the reaction mixture may be conducted with known methods in the art.

When the cooling of the reaction mixture is started, the temperature of the reaction mixture may be the same, higher, or lower than the temperature of the reaction mixture in the beginning of the process.

In some embodiments, cooling of the reaction mixture is continued so that when the acrylamide concentration reaches 37 wt % to 55 wt %, the temperature of the reaction mixture is within a range of 10° C. to 18° C., or 10° C. to 21° C. In other words, the time period of the cooling of the reaction mixture to the temperature of 10° C. to 18° C., or 10° C. to 21° C., is the time period when the acrylamide concentration of at least 27 wt % (more specifically 27 wt % to 38 wt %) increases to acrylamide concentration 37 wt % to 55 wt % (more specifically 40 wt % to 50 wt %).

In one embodiment, the cooling of the reaction mixture is continued so that so that when the acrylamide concentration reaches 37 wt % to 55 wt %, the temperature is within a range of 10° C. to 16° C., more specifically 13° C. to 16° C., and even more specifically, the temperature is 15° C. In one embodiment the cooling is started, for example, after the reaction mixture has been maintained at 15° C. to 25° C. In one embodiment the reaction mixture is cooled by at least 10° C., more specifically at least 5° C., even more specifically at least 4° C. The cooling may be conducted linearly or stepwise, typically linearly.

In one embodiment, the reaction mixture is maturated at a temperature within a range of 10° C. to 18° C., or 10° C. to 21° C. when the acrylamide concentration reaches 37 wt % to 55 wt %.

During the maturation, substantially no acrylonitrile, and more specifically no acrylonitrile, is fed to the reactor. During the maturation, unreacted acrylonitrile in the reactor reacts to acrylamide. Maturation begins after the reaction mixture has been cooled and the temperature of the reaction mixture is within the range of 10° C. to 18° C., or 10° C. to 21° C., and/or after the feeding of acrylonitrile into the reactor has ended. More specifically the maturation is continued until final concentration of the acrylonitrile in the reaction mixture is at most 1000 ppm, at most 500 ppm, at most 250 ppm, at most 100 ppm, at most 50 ppm, at most 10 ppm, or at most 0 ppm.

In one embodiment of the process, the temperature of the reaction mixture is maintained at 15° C. to 25° C. for 30 min to 90 min, such as for 45 min to 60 min and the cooling of the reaction mixture to the temperature of 10° C. to 18° C., or 10° C. to 21° C. is performed during a period of time of 45 min to 120 min, such as 60 min to 120 min.

After reaction completion the biocatalyst may be separated from the aqueous AMD-solution by any known separation method e.g., centrifugation, flotation, filter-pressing or filtration. The aqueous acrylamide solution produced by the process is then typically used in manufacturing of polyacrylamide.

The separation or harvest of the biomass cells after fermentation may be in some exemplary embodiments be facilitated by the addition of flocculants to help collect the cells and may be conducted prior to the separation method, e.g., before centrifugation/filtration. In exemplary embodiments a salt or other stabilizer will be added before freezing the biomass. For example after the cells have been harvested a suitable preservative salt may be added to the biomass/cell mass, e.g., an ammonium, calcium, iron, magnesium, potassium or sodium salt.

In some exemplary embodiments the separation or harvest of the biomass cells after fermentation can take place after first adding a cationic solution, followed by the addition of an anionic solution. The amount of cationic solution added can range e.g., from 2 to 10% with respect to the mass to be flocculated, while the anionic solution can range e.g., from 0.5 to 3.5%. In some exemplary embodiments, the cationic solution ranges from 4 to 7%, while the anionic solution ranges from 1 to 2%. Also, the suspension may be stirred during this process. [In this process the low molecular weight cationic flocculant acts as a coagulant resulting in the formation of a micro flocculant and the anionic solution is subsequently added and the micro flocculant serves as a substrate providing for the agglomeration of the high molecular weight anionic flocculant].

A time after the addition of the anionic solution, e.g., about 5-30, 5-15 or 10 minutes, a flocculant is formed which can be separated by a desired means, e.g., by centrifugation, flotation, filter-pressing or filtration. For example, separation can be effected by the use of filters having pores with a diameter <0.45 pm. If desired, the filtered biomass can be resuspended, optionally in demineralized water and filtration repeated providing for further removal of the broth culture residues from the biomass.

As noted above, if the resultant recovered biomass is stored as a paste, the biomass is usually mixed with a stabilizer or preservative, e.g., an ammonium, calcium, iron, magnesium, potassium or sodium salt.

Having described the invention in detail the invention is further described in the following examples.

### EXAMPLES

The following examples are presented for illustrative purposes only and are not intended to be limiting.

### Example 1: Production of Variant Nitrile Hydratase and Variant Nitrile Hydratase Operon

The development of the inventive biocatalyst included screening of multiple nitrile hydratases and host cells, introducing various genetic modifications into numerous nitrile hydratase genes and evaluation of the performance (stability/activity) of the variants. Nitrile hydratases originating from several different microbe species were studied. Methods used by the inventors for the improvement of the biocatalyst included in vivo recombination, site specific and random mutagenesis, chaperone co-expression, operon optimization, expression plasmid optimization, codon usage optimization and fermentation optimization.

These efforts resulted in the isolation of variant nitrile hydratase alpha and beta subunits respectively comprising SEQ ID NO: 2 and 1. Both of these variants were derived from the alpha and beta subunits of an nitrile hydratase produced by a Pseudonocardia thermophila strain (Pseudonocardia thermophila strain available under deposit DSM 43832). The specific combination of mutations comprised in these variants is further shown in the sequence alignments in FIGS. 1 and 2. These variants when co-expressed were found to yield an enzyme exhibiting the best combination of enhanced stability and/or activity compared to the parental nitrile hydratase endogenously produced by the Pseudonocardia thermophila strain.

Also, the inventors observed that a variant nitrile hydratase comprising an alpha unit comprising L6T, A19V, F126Y and a beta subunit comprising E108R/A200E/S212Y mutations possessed the second best combination of enhanced stability and/or activity compared to the parental nitrile hydratase.

Additionally, further in order to enhance nitrile hydratase expression and nitrile hydratase stability during amide synthesis (when introduced into an exemplary industrial host strain) the endogenous nitrile hydratase gene containing operon of Pseudonocardia thermophila DSM 43832 was further extensively modified in relation to the wild-type nitrile hydratase genes and operon containing. Specifically, as shown in FIG. 3, the codon usage of the variant nhhA and nhhB genes and that of the nhhG gene was optimized for C. glutamicum (based on the codon usage of highly expressed genes in C. glutamicum as published in Eikmanns, B. (1998), Identification, Sequence Analysis, and Expression of a Corynebacterium glutamicum Gene Cluster Encoding the Three Glycolytic Enzymes Glyceraldehyde-3-Phosphate Dehydrogenase, 3-Phosphoglycerate Kinase, and Triosephosphate Isomerase, J Bac 174, 6076-6086); the nhhBAG operon was modified to include NdeI/XhoI restriction sites used for cloning of the DSM 43832 strain nhhBAG operon, the operon was modified to remove the amidase gene present in the endogenous operon upstream of nhhB; and the operon was modified to include intergenic ribosomal binding sites to eliminate the overlap because in the wildtype operon sequence the TGA stop codon of nhhB overlaps with the ATG start codon of nhhA and similarly the TGA stop codon of nhhA overlaps with the GTG start codon of nhhG. The variant operon comprising these modifications is contained in SEQ ID NO: 7.

### Example 2: Development of Novel Nitrile Hydratase and Operon

The development of the inventive biocatalyst Corynebacterium glutamicum strain comprised introducing plasmids comprising the operon described in the previous example into a selected industrial strain. As previously noted the inventors selected as the industrial parental strain C. glutamicum MB001(DE3) which has been deposited in the German Collection of Microorganisms and Cell Cultures (DSMZ) under strain No. 102071. Also, a detailed description of this strain is published in Kortmann M. et al. (2015), “A chromosomally encoded T7 RNA polymerase-dependent gene expression system for Corynebacterium glutamicum: construction and comparative evaluation at the single-cell level”, Microb Biotechnol. 8:253-65.

The parental strain was selected as it has been used in other industrial processes and because it contains no nitrile hydratase genes. An exemplary production strain comprises the nitrile hydratase operon present on a plasmid with approximately 30 copies per cell. However, it is anticipated that comparable or further enhanced results may be achieved by instead engineering the C. glutamicum MB001(DE3) strain such that one or more copies of the nitrile hydratase operon are integrated into the C. glutamicum cell genome (i.e., for improved strain stability and to avoid the use of antibiotic resistance genes and in order to further maximize the enzyme yield).

Additionally, since the use of encapsulated nitrile hydratase from Pseudonocardia thermophila has been described (see e.g., Martinez et al. (2014), “Acrylamide production using encapsulated nitrile hydratase from Pseudonocardia thermophila in a sol-gel matrix”, J Molecular Catalysis B: Enzymatic, 100, 19-24) and reportedly led to improved thermal stability compared to the enzyme in solution, alternatively the variant nitrile hydratase enzyme may be encapsulated in a sol-gel matric or lipid vesicle and used in this form as a biocatalyst in amide synthesis processes).

### Example 3: Use of Novel Nitrile Hydratase Biocatalyst for AMD Synthesis

The performance of exemplary biocatalysts comprising different variant nitrile hydratases expressed in different bacteria including the C. glutamicum nitrile hydratase producing production strain described in the previous example were compared for their stability/activity when used during synthesis of Acrylamide (AMD) from Acrylonitrile (AN) as described below. These experiments and the results are further summarized in the tables below.

In these experiments demineralized water and biocatalyst were mixed and AN feed was started. As shown, in these methods the entire AN feed was fed in either 3 hours or in 1 hour. In these syntheses the targeted AMD-concentration was either 52 or 54%. As a result of the sampling during the synthesis the final AMD-concentrations was usually a little smaller. If the synthesis was successful, no AN was detected from the solution at test end.

Biocatalyst X1 has the variant nitrile hydratase enzyme derived from Pseudonocardia and subsequently modified to comprise specific mutations and the host is E. coli. It converted all AN to AMD at a dosage of 5.7 g dry cells/kg 100% AMD. However the separation of the biocatalyst from the ready AMD with centrifugation was not successful, thus E. coli was discarded as the host cell.

As noted above, different biocatalysts comprising different variant nitrile hydratases (also derived from Pseudonocardia and subsequently modified to comprise specific mutations) were expressed in different bacteria including the C. glutamicum nitrile hydratase producing production strain described in the previous example and were compared for their stability/activity when used during synthesis of Acrylamide (AMD) from Acrylonitrile (AN).

The experiments and the results attained thereby are summarized in the Table 1 and Table 2 below. Also, the different biocatalysts identified in the tables and their comparative results when utilized for AMD synthesis are briefly summarized below:

Biocatalyst X2 comprises the nitrile hydratase enzyme from Rhodococcus and the host is Corynebacterium glutamicum. It converted only about one third of the added AN, resulting at a final AMD-concentration of 18% at a dosage of 6.0 g dry cells/kg100% AMD. Thus development of this combination was discontinued.

Biocatalysts X3, X4 and X5 comprise the nitrile hydratase enzyme from Pseudonocardia and the host is Corynebacterium glutamicum. They are all different development phase biocatalysts from different batches. All 3 versions converted all AN to AMD in the experiments.

Biocatalyst X4 was dried after fermentation and before it was used in the AMD synthesis it was re-wetted. The re-wetted biocatalyst was able to convert all AN to AMD.

Biocatalyst X5 comprises the final version, as shown in the Tables it exhibited substantially higher activity per cell dry mass. A dosage of only 1.7 g dry cells/kg 100% AMD converted all AN to AMD.

Biocatalyst R1 comprises the nitrile hydratase from Rhodococcus and the host is Rhodococcus. As shown in the Tables, in an experiment where all the feed AN was added in 1 h, it converted only about ⅔ of the added AN to AMD at the high dosage of 6.9 kg dry cells/kg 100% AMD. The biocatalyst is deactivated by the high levels of AN.

Biocatalyst X6 comprises the nitrile hydratase enzyme from Pseudonocardia and the host is Corynebacterium glutamicum. In an experiment where all AN was added in 1 h, it converted all added AN to AMD at the dosage of 4.6 kg dry cells/kg 100% AMD in the 21 h experiment. This biocatalyst thus tolerates high AN and AMD concentrations.

The results observed from these experiments are summarized in the table below.

TABLE 1

| | | | | Acrylonitrile | | | | |
|---|---|---|---|---|---|---|---|---|
| | | | | concentration during synthesis | | | | |
| | | | | | | | Biocat- | |
| | Acrylamide | | | | | | alyst | |
| | synthesis test setup | | | Produced | | AN feed | dosage | AN |
| | | | Nitrile | AMD | AN | rate | [g dry | concen- |
| | | | Hydratase | concen- | feed | [% of | cells/ | tration |
| | Biocat- | Host | origin | tration | time | total | kg100% | at 1.5 h |
| Remark | alyst | species | species | [%] | [h] | per hour] | AMD] | [%] |
| | X1 | Escherichia | Pseudono- | 52 | 3 | 47- | 5.7 | 2.09 |
| | | coli | cardia | | | 33- | | |
| | | | | | | 20 | | |
| | X2 | Coryne- | Rhodococcus | 52 | 3 | 47- | 6.0 | 9.97 |
| | | bacterium | | | | 33- | | |
| | | glutamicum | | | | 20 | | |
| | X3 | Coryne- | Pseudono- | 52 | 3 | 47- | 5.4 | 0.78 |
| | | bacterium | cardia | | | 33- | | |
| | | glutamicum | | | | 20 | | |
| Dried | X4 | Coryne- | Pseudono- | 52 | 3 | 33- | 5.3 | 0.59 |
| biocat- | | bacterium | cardia | | | 33- | | |
| alyst | | glutamicum | | | | 33 | | |
| Final | X5 | Coryne- | Pseudono- | 54 | 3 | 53- | 1.7 | 0.39 |
| version | | bacterium | cardia | | | 29- | | |
| | | glutamicum | | | | 18 | | |
| | R1 | Rhodococcus | Rhodococcus | 52 | 1 | 100 | 6.9 | 13.92 |
| | X6 | Coryne- | Pseudono- | 52 | 1 | 100 | 4.6 | 9.17 |
| | | bacterium | cardia | | | | | |
| | | glutamicum | | | | | | |

| | | Acrylamide | | | | | | |
|---|---|---|---|---|---|---|---|---|
| | | concentration during synthesis | | | | | | |
| | Acrylamide | AN | AN | AN | AMD | AMD | AMD | AMD |
| | synthesis | concen- | concen- | concen- | concen- | concen- | concen- | concen- |
| | test setup | tration | tration | tration | tration | tration | tration | tration |
| | Biocat- | at 3 h | at 5 h | at 21 h | at 1.5 h | at 3 h | at 5 h | at 21 h |
| Remark | alyst | [%] | [%] | [%] | [%] | [%] | [%] | [%] |
| | X1 | 4.04 | 0 | 0 | 36.05 | 45.07 | 51.6 | 51.04 |
| | X2 | 10.88 | 10.99 | 10.95 | 18.56 | 18.66 | 18.22 | 18.29 |
| | X3 | 2.32 | 0 | 0 | 36.99 | 48.17 | 50.95 | 50.65 |
| Dried | X4 | 4.02 | 0 | 0 | 33.67 | 44.91 | 50.66 | 50.62 |
| biocat- | | | | | | | | |
| alyst | | | | | | | | |
| Final | X5 | 1.75 | 0 | 0 | 41.51 | 51.2 | 53.77 | 53.99 |
| version | | | | | | | | |
| | R1 | 13.74 | 13.87 | 13.56 | 31.83 | 32.28 | 31.46 | 31.72 |
| | X6 | 4.38 | 1.7 | 0 | 38.45 | 45.12 | 48.36 | 50.64 |

Based on the results in Table 1 above it can be seen that the inventive biocatalyst possesses enhanced stability and activity over prolonged time (at least 3 hours) when utilized for AMD synthesis. Also the results show that different forms of the inventive biocatalyst may be used, e.g., dried forms.

Exemplary sequences used in the examples are contained in the Sequence Listing on the following page.

SEQUENCE LISTING

Variant DSM 43832 nhhB beta subunit: amino acid sequence: SEQ ID NO: 1
MNGVYDVGGTDGLGPINRPADEPVFRAEWEKVAFAMFPATFRAGFMGLDEFRFGIEQMNPAEYLESPYY
WHWIRTYIHHGVRTGKIDLEELERRTQYYRENPDAPLPDHEQKPELIEFVNQAVYGGLPASREVDRPPKFKEG
DVVRFSTASPKGHARRARYVRGKTGTVVKHHGAYIYPDTAGNGLGECPEHLYTVRFTEQELWGPEGDPNSS
VYYDCWEPYIELVDTKAAAA*
Variant DSM 43832 nhhA alpha subunit: amino acid sequence: SEQ ID NO: 2
MTENITRKSDEEIQKEITVRVKALESMLIEQGILTTSMIDRMAEIYENEVGPHLGAKVVVKAWTDPEFKKRLLA
DGTEACKELGIGGLQGEDMMWVENTDEVHHVVVCTLCSCYPWPVLGLPPNWYKEPQYRSRVVREPRQLL
KEEFGFEVPPSKEIKVWDSSSEMRFVVLPQRPAGTDGWSEEELATLVTRESMIGVEPAKAVA*
DSM 43832 nhhG activator protein: amino acid sequence: SEQ ID NO: 3
MSAEAKVRLKHCPTAEDRAAADALLAQLPGGDRALDRGFDEPWQLRAFALAVAACRAGRFEWKQLQQALI
SSIGEWERTHDLDDPSWSYYEHFVAALESVLGEEGIVEPEALDERTAEVLANPPNKDHHGPHLEPVAVHPAV
RS*
Variant DSM 43832 nhhB: beta subunit: Nucleic acid coding sequence SEQ ID NO: 4
ATGAACGGCGTTTACGACGTTGGCGGCACCGACGGCCTGGGTCCAATCAACCGCCCAGCAGACGAGCC
AGTTTTCCGCGCTGAGTGGGAGAAGGTTGCATTCGCTATGTTCCCAGCAACCTTCCGCGCTGGCTTCATG
GGCCTGGACGAGTTCCGCTTCGGCATCGAGCAGATGAACCCAGCAGAGTACCTGGAGTCCCCATACTAC
TGGCACTGGATCCGCACCTACATCCACCACGGCGTTCGCACCGGCAAGATCGACCTGGAGGAGCTGGA
GCGTCGCACCCAGTACTACCGCGAGAACCCAGACGCTCCACTGCCAGACCACGAGCAGAAGCCAGAGC
TGATCGAGTTCGTTAACCAGGCAGTTTACGGCGGCCTGCCAGCTTCCCGCGAGGTTGACCGCCCACCAA
AGTTCAAGGAAGGCGACGTTGTTCGCTTCTCCACCGCATCCCCAAAGGGTCACGCACGCCGCGCTCGCT
ACGTTCGCGGCAAGACCGGCACCGTTGTTAAGCACCACGGCGCATACATCTACCCAGACACCGCTGGTA
ACGGCCTGGGCGAGTGCCCAGAGCACCTGTACACCGTTCGCTTCACCGAGCAGGAGCTGTGGGGTCCA
GAGGGCGACCCAAACTCCTCCGTTTACTACGACTGCTGGGAGCCATACATCGAGCTGGTTGACACCAAG
GCAGCTGCAGCTTGA
Variant DSM 43832 nhhA alpha subunit: Nucleic acid coding sequence: SEQ ID NO: 5
ATGACCGAGAACATCACCCGCAAGTCCGACGAGGAGATCCAGAAGGAGATCACCGTTCGCGTTAAGGC
TCTGGAGTCCATGCTGATCGAGCAGGGCATCCTGACCACCTCTATGATCGACCGCATGGCAGAGATCTA
CGAGAACGAGGTTGGCCCACACCTGGGCGCTAAGGTTGTTGTTAAGGCATGGACCGACCCAGAGTTCA
AGAAGCGCCTGCTGGCTGACGGTACCGAGGCATGCAAGGAGCTGGGTATCGGCGGCCTGCAGGGCGA
GGACATGATGTGGGTTGAGAACACCGACGAGGTTCACCACGTTGTTGTTTGCACCCTGTGCTCCTGCTAC
CCATGGCCAGTTCTGGGCCTGCCACCAAACTGGTACAAGGAGCCACAGTACCGCTCCCGCGTTGTTCGC
GAGCCACGCCAGCTGCTGAAGGAAGAGTTCGGCTTCGAGGTTCCACCATCCAAGGAGATCAAGGTTTG
GGACTCCTCCTCCGAGATGCGCTTCGTTGTTCTGCCACAGCGCCCAGCTGGTACCGACGGTTGGTCCGAA
GAGGAGCTGGCAACCCTGGTTACCCGCGAGTCCATGATCGGCGTTGAGCCAGCAAAGGCTGTTGCCTG
A
Variant DSM 43832 nhhG activator protein: Nucleic acid coding sequence: SEQ ID NO: 6
ATGTCCGCTGAGGCAAAGGTTCGCCTGAAGCACTGCCCAACCGCAGAGGACCGCGCAGCTGCAGACGC
ACTGCTGGCTCAGCTGCCAGGCGGCGACCGCGCACTGGACCGCGGCTTCGACGAGCCATGGCAGCTGC
GCGCTTTCGCACTGGCTGTTGCAGCATGCCGCGCAGGCCGCTTCGAGTGGAAGCAGCTGCAGCAGGCTC
TGATCTCCTCCATCGGCGAGTGGGAGCGCACCCACGACCTGGACGACCCATCCTGGTCCTACTACGAGC
ACTTCGTTGCTGCACTGGAGTCCGTTCTGGGCGAGGAAGGCATCGTTGAGCCAGAGGCACTGGACGAG
CGCACCGCAGAGGTTCTGGCTAACCCACCAAACAAGGACCACCACGGCCCACACCTGGAGCCAGTTGCA
GTTCACCCAGCTGTTCGCTCCTAA
Variant DSM 43832 nhhBAG operon (including NdeI/XhoI restriction sites used for cloning DSM
43832 Strain nhhBAG operon and lacking the amidase gene present in endogenous operon
upstream of nhhB): SEQ ID NO: 7
CATATGAACGGCGTGTACGACGTTGGTGGCACCGATGGTCTGGGTCCGATTAACCGTCCGGCGGATGA
GCCGGTGTTCCGTGCGGAGTGGGAAAAGGTTGCGTTCGCGATGTTTCCGGCGACCTTCCGTGCGGGCTT
TATGGGTCTGGATGAGTTCCGTTTTGGTATTGAACAGATGAACCCGGCGGAGTACCTGGAAAGCCCGTA
CTATTGGCACTGGATCCGTACCTATATTCACCACGGCGTGCGTACCGGCAAGATCGACCTGGAGGAACT
GGAGCGTCGTACCCAATACTATCGTGAAAACCCGGATGCGCCGCTGCCGGATCATGAACAGAAACCGG
AGCTGATTGAATTCGTGAACCAGGCGGTTTATGGTGGCCTGCCGGCGAGCCGTGAGGTGGACCGTCCG
CCGAAGTTCAAAGAAGGCGATGTGGTTCGTTTTAGCACCGCGAGCCCGAAGGGTCATGCGCGTCGTGC
GCGTTATGTTCGTGGCAAGACCGGTACCGTGGTTAAACACCACGGTGCGTACATCTATCCGGACACCGC
GGGTAACGGCCTGGGCGAGTGCCCGGAACACCTGTACACCGTTCGTTTTACCGAACAAGAACTGTGGG
GTCCGGAGGGTGACCCGAACAGCAGCGTGTACTATGATTGCTGGGAGCCGTATATTGAACTGGTTGATA
CCAAAGCGGCGGCGGCGTGAAAGGAGATATAGATATGACCGAAAACATCACCCGTAAGAGCGACGAG
GAAATCCAGAAAGAGATTACCGTGCGTGTTAAGGCGCTGGAAAGCATGCTGATCGAGCAAGGTATTCT
GACCACCAGCATGATCGATCGTATGGCGGAAATTTACGAAAACGAAGTGGGTCCGCACCTGGGTGCGA
AGGTGGTTGTGAAAGCGTGGACCGACCCGGAGTTCAAGAAACGTCTGCTGGCGGATGGCACCGAAGC
GTGCAAAGAGCTGGGTATTGGTGGCCTGCAGGGCGAAGACATGATGTGGGTGGAAAACACCGATGAG
GTTCACCACGTTGTGGTTTGCACCCTGTGCAGCTGCTATCCGTGGCCGGTTCTGGGTCTGCCGCCGAACT
GGTACAAAGAACCGCAGTATCGTAGCCGTGTGGTTCGTGAGCCGCGTCAACTGCTGAAAGAGGAGTTC
GGCTTTGAAGTGCCGCCGAGCAAGGAGATCAAAGTTTGGGACAGCAGCAGCGAGATGCGTTTTGTGGT
TCTGCCGCAACGTCCGGCGGGTACCGATGGTTGGAGCGAAGAGGAGCTGGCGACCCTGGTGACCCGTG
AAAGCATGATTGGTGTGGAGCCGGCGAAGGCGGTTGCGTGAAAGGAGATATAGATATGAGCGCGGAG
GCGAAAGTGCGTCTGAAACACTGCCCGACCGCGGAAGATCGTGCGGCGGCGGATGCGCTGCTGGCGCA
GCTGCCGGGTGGCGACCGTGCGCTGGATCGTGGTTTCGACGAGCCGTGGCAACTGCGTGCGTTTGCGC
TGGCGGTTGCGGCGTGCCGTGCGGGTCGTTTCGAATGGAAGCAGCTGCAGCAAGCGCTGATCAGCAGC
ATTGGCGAGTGGGAACGTACCCACGATCTGGACGATCCGAGCTGGAGCTACTATGAGCACTTTGTGGC
GGCGCTGGAAAGCGTTCTGGGCGAGGAAGGCATCGTGGAGCCGGAAGCGCTGGATGAGCGTACCGCG
GAAGTTCTGGCGAACCCGCCGAACAAAGACCACCACGGCCCGCACCTGGAGCCGGTGGCGGTTCACCC
GGCGGTGCGTAGCTAACTCGAG
Wildtype DSM 43832 nhhBAG operon (lacking the amidase gene present in endogenous operon
upstream of nhhB): SEQ ID NO: 8
ATGAACGGCGTGTACGACGTCGGCGGCACCGATGGGCTGGGCCCGATCAACCGGCCCGCGGACGAACC
GGTCTTCCGCGCCGAGTGGGAGAAGGTCGCGTTCGCGATGTTCCCGGCGACGTTCCGGGCCGGCTTCAT
GGGCCTGGACGAGTTCCGGTTCGGCATCGAGCAGATGAACCCGGCCGAGTACCTCGAGTCGCCGTACT
ACTGGCACTGGATCCGCACCTACATCCACCACGGCGTCCGCACCGGCAAGATCGATCTCGAGGAGCTGG
AGCGCCGCACGCAGTACTACCGGGAGAACCCCGACGCCCCGCTGCCCGAGCACGAGCAGAAGCCGGAG
TTGATCGAGTTCGTCAACCAGGCCGTCTACGGCGGGCTGCCCGCAAGCCGGGAGGTCGACCGACCGCC
CAAGTTCAAGGAGGGCGACGTGGTGCGGTTCTCCACCGCGAGCCCGAAGGGCCACGCCCGGCGCGCGC
GGTACGTGCGCGGCAAGACCGGGACGGTGGTCAAGCACCACGGCGCGTACATCTACCCGGACACCGCC
GGCAACGGCCTGGGCGAGTGCCCCGAGCACCTCTACACCGTCCGCTTCACGGCCCAGGAGCTGTGGGG
GCCGGAAGGGGACCCGAACTCCAGCGTCTACTACGACTGCTGGGAGCCCTACATCGAGCTCGTCGACAC
GAAGGCGGCCGCGGCATGACCGAGAACATCCTGCGCAAGTCGGACGAGGAGATCCAGAAGGAGATCA
CGGCGCGGGTCAAGGCCCTGGAGTCGATGCTCATCGAACAGGGCATCCTCACCACGTCGATGATCGACC
GGATGGCCGAGATCTACGAGAACGAGGTCGGCCCGCACCTCGGCGCGAAGGTCGTCGTGAAGGCCTG
GACCGACCCGGAGTTCAAGAAGCGTCTGCTCGCCGACGGCACCGAGGCCTGCAAGGAGCTCGGCATCG
GCGGCCTGCAGGGCGAGGACATGATGTGGGTGGAGAACACCGACGAGGTCCACCACGTCGTCGTGTG
CACGCTCTGCTCCTGCTACCCGTGGCCGGTGCTGGGGCTGCCGCCGAACTGGTTCAAGGAGCCGCAGTA
CCGCTCCCGCGTGGTGCGTGAGCCCCGGCAGCTGCTCAAGGAGGAGTTCGGCTTCGAGGTCCCGCCGA
GCAAGGAGATCAAGGTCTGGGACTCCAGCTCCGAGATGCGCTTCGTCGTCCTCCCGCAGCGCCCCGCGG
GCACCGACGGGTGGAGCGAGGAGGAGCTCGCCACCCTCGTCACCCGCGAGTCGATGATCGGCGTCGAA
CCGGCGAAGGCGGTCGCGTGAGCGCCGAGGCGAAGGTCCGCCTGAAGCACTGCCCCACGGCCGAGGA
CCGGGCGGCGGCCGACGCGCTGCTCGCGCAGCTGCCCGGCGGCGACCGCGCGCTCGACCGCGGCTTCG
ACGAGCCGTGGCAGCTGCGGGCGTTCGCGCTGGCGGTCGCGGCGTGCAGGGCGGGCCGGTTCGAGTG
GAAGCAGCTGCAGCAGGCGCTGATCTCCTCGATCGGGGAGTGGGAGCGCACCCACGATCTCGACGATC
CGAGCTGGTCCTACTACGAGCACTTCGTCGCCGCGCTGGAATCCGTGCTCGGCGAGGAAGGGATCGTC
GAGCCGGAGGCGCTGGACGAGCGCACCGCGGAGGTCTTGGCCAACCCGCCGAACAAGGATCACCATG
GACCGCATCTGGAGCCCGTCGCGGTCCACCCGGCCGTGCGGTCCTGA

Having described exemplary embodiments of the invention, the invention is further described in the claims which follow.

What is claimed is:

1. A variant nitrile hydratase comprising (i) an alpha subunit (nhhA) having an amino acid sequence which possesses at least 98, 99 or 100% sequence identity to SEQ ID NO: 2 and (ii) a beta subunit (nhhB) comprising an amino acid sequence which possesses at least 98, 99 or 100% sequence identity to SEQ ID NO: 1, with the proviso that the alpha subunit comprises one, two or three of the following mutations: L6T, A19V and F126Y and the beta subunit comprises one or both of the following mutations: E108D and A200E or comprises all three of the following mutations E108R, A200E and S212Y;
wherein said variant nitrile hydratase possesses enhanced stability and/or activity.

2. The nitrile hydratase of claim 1, wherein:
(i) the alpha subunit comprises the following mutations: L6T, A19V and F126Y and the beta subunit comprises the following mutations: E108D and A200E or E108R, A200E and S212Y; or
(ii) said enzyme subunits are expressed in association with an nhhG activator protein, optionally one comprising an amino acid sequence which is at least 98, 99 or 100% sequence identical to SEQ ID NO: 3;
(iii) the nitrile hydratase comprises a soluble enzyme or cell which produces the nitrile hydratase;
(iv) the nitrile hydratase is immobilized to a solid support;
(v) the nitrile hydratase is encapsulated, e.g., in a vesicle, sol-gel matrix, or other material that optionally provides for improved thermal stability compared to the enzyme in solution; or
(vi) any combination of (i) to (v).

3. A nucleic acid or nucleic acids which encode for an nitrile hydratase comprising an alpha subunit and beta subunit and optionally an activator protein according to claim 1, wherein the nucleic acids encoding one or more of the alpha subunit, beta subunit and optionally the activator protein are codon optimized to increase expression in a desired microorganism, optionally a yeast, fungus or bacterium or further optionally the nucleic acids encoding one or more of the alpha subunit, beta subunit and optionally the activator protein are codon optimized to increase expression in C. glutamicum ATCC13032 or its derivative MB001(DE3) which has deposited in the German Collection of Microorganisms and Cell Cultures (DSMZ) under strain No. 102071.

4. The nucleic acid or nucleic acids of claim 3, wherein
(i) the desired microorganism is a bacterium selected from Rhodococcus, Aspergillus, Acidovorax, Agrobacterium, Bacillus, Bradyrhizobium, Brevibacterium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus or the microorganism may be selected from bacteria of the genus Rhodococcus, Pseudomonas, Escherichia and Geobacillus or is selected from the following species Rhodococcus rhodochrous, Rhodococcus pyridinovorans, Rhodococcus erythropolis, Rhodococcus equi, Rhodococcus ruber, Rhodococcus opacus, Aspergillus niger, Acidovorax avenae, Acidovorax facilis, Agrobacterium tumefaciens, Agrobacterium radiobacter, Bacillus subtilis, Bacillus pallidus, Bacillus smithii, Bacillus sp BR449, Bradyrhizobium oligotrophicum, Bradyrhizobium diazoefficiens, Bradyrhizobium japonicum, Burkholderia cenocepacia, Burkholderia gladioli, Escherichia coli, Geobacillus sp. RAPc8, Klebsiella oxytoca, Klebsiella pneumonia, Klebsiella variicola, Mesorhizobium ciceri, Mesorhizobium opportunistum, Mesorhizobium sp F28, Moraxella, Pantoea endophytica, Pantoea agglomerans, Pseudomonas chlororaphis, Pseudomonas putida, Rhizobium, Rhodopseudomonas palustris, Serratia liquefaciens, Serratia marcescens, Amycolatopsis, Arthrobacter, Brevibacterium sp CH1, Brevibacterium sp CH2, Brevibacterium sp R312, Brevibacterium imperiale, Corynebacterium nitrilophilus, Corynebacterium pseudodiphteriticum, Corynebacterium glutamicum, Corynebacterium hoffmanii, Microbacterium imperiale, Microbacterium smegmatis, Micrococcus luteus, Nocardia globerula, Nocardia rhodochrous, Pseudonocardia thermophila, Trichoderma, Myrothecium verrucaria, Aureobasidium pullulans, Candida famata, Candida guilliermondii, Candida tropicalis, Cryptococcus flavus, Cryptococcus sp UFMG-Y28, Debaryomyces hanseii, Geotrichum candidum, Geotrichum sp JR1, Hanseniaspora, Kluyveromyces thermotolerans, Pichia kluyveri, Rhodotorula glutinis, Comomonas testosterone, Pyrococcus abyssi, Pyrococcus furiosus, Pyrococcus horikoshii, Brevibacterium casei, or Nocardia sp. 163; and optionally the microorganism comprises a bacterium of the species Corynebacterium glutamicum, further optionally strain C. glutamicum ATCC13032 or its derivative MB001(DE3);
(ii) the beta subunit is encoded by the nucleic acid of SEQ ID NO: 4;
(iii) the alpha subunit is encoded by the nucleic acid of SEQ ID NO: 5;
(iv) the activator protein is encoded by the nucleic acid of SEQ ID NO:6; or
(v) any combination of (i) to (iv).

5. A nitrile hydratase operon comprising nucleic acids which encode for the alpha (nhhA) and beta (nhhB) subunits of a variant nitrile hydratase and optionally an activator protein (nhhG) according to claim 1.

6. The operon of claim 5, which is derived from a yeast, fungus or bacterium that expresses nitrile hydratase, optionally a Pseudomonas bacterium, further optionally Pseudonocardia thermophila.

7. The operon of claim 5, which comprises SEQ ID NO: 7.

8. An extrachromosomal sequence, optionally a plasmid comprising a nucleic acid or operon according to claim 3.

9. A microorganism, optionally a yeast, fungus or bacterium, further optionally an industrial microorganism which optionally does not endogenously express nitrile hydratase or which endogenously expresses nitrile hydratase, which microorganism is engineered to comprise nucleic acids encoding a nitrile hydratase according to claim 1, or nucleic acids encoding for said nitrile hydratase or an operon or extrachromosomal sequence comprising said nucleic acids.

10. The microorganism according to claim 9, is a bacterium selected from Rhodococcus, Aspergillus, Acidovorax, Agrobacterium, Bacillus, Bradyrhizobium, Brevibacterium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus or is selected from bacteria of the genus Rhodococcus, Pseudomonas, Escherichia and Geobacillus or is selected from the following species Rhodococcus rhodochrous, Rhodococcus pyridinovorans, Rhodococcus erythropolis, Rhodococcus equi, Rhodococcus ruber, Rhodococcus opacus, Aspergillus niger, Acidovorax avenae, Acidovorax facilis, Agrobacterium tumefaciens, Agrobacterium radiobacter, Bacillus subtilis, Bacillus pallidus, Bacillus smithii, Bacillus sp BR449, Bradyrhizobium oligotrophicum, Bradyrhizobium diazoefficiens, Bradyrhizobium japonicum, Burkholderia cenocepacia, Burkholderia gladioli, Escherichia coli, Geobacillus sp. RAPc8, Klebsiella oxytoca, Klebsiella pneumonia, Klebsiella variicola, Mesorhizobium ciceri, Mesorhizobium opportunistum, Mesorhizobium sp F28, Moraxella, Pantoea endophytica, Pantoea agglomerans, Pseudomonas chlororaphis, Pseudomonas putida, Rhizobium, Rhodopseudomonas palustris, Serratia liquefaciens, Serratia marcescens, Amycolatopsis, Arthrobacter, Brevibacterium sp CH1, Brevibacterium sp CH2, Brevibacterium sp R312, Brevibacterium imperiale, Corynebacterium nitrilophilus, Corynebacterium pseudodiphteriticum, Corynebacterium glutamicum, Corynebacterium glutamicum, Corynebacterium hoffmanii, Microbacterium imperiale, Microbacterium smegmatis, Micrococcus luteus, Nocardia globerula, Nocardia rhodochrous, Pseudonocardia thermophila, Trichoderma, Myrothecium verrucaria, Aureobasidium pullulans, Candida famata, Candida guilliermondii, Candida tropicalis, Cryptococcus flavus, Cryptococcus sp UFMG-Y28, Debaryomyces hanseii, Geotrichum candidum, Geotrichum sp JR1, Hanseniaspora, Kluyveromyces thermotolerans, Pichia kluyveri, Rhodotorula glutinis, Comomonas testosterone, Pyrococcus abyssi, Pyrococcus furiosus, Pyrococcus horikoshii, Brevibacterium casei, or Nocardia sp. 163, and optionally is a bacterium of the species Corynebacterium glutamicum, optionally strain C. glutamicum ATCC13032 or its derivative MB001(DE3).

11. The microorganism of claim 9, which is an industrial microorganism which endogenously expresses nitrile hydratase, and said nucleic acids or operon replaces the endogenous nitrile hydratase gene or operon comprising the endogenous nitrile hydratase gene.

12. A method for producing an amide compound from a nitrile compound, the method comprising: contacting the nitrile compound with an nitrile hydratase or a microbe which expresses said nitrile hydratase according to claim 1.

13. The method of claim 12, wherein the amide compound is selected from the group consisting of acrylamide, methacrylamide, acetamide, and nicotinamide and preferably is acrylamide, and the nitrile compound is selected from the group consisting of acrylonitrile, methacrylonitrile, acetonitrile, and 3-cyanopyridine and preferably is acrylonitrile.

14. The method of claim 12 which uses one or more of a soluble nitrile hydratase, an encapsulated nitrile hydratase, an immobilized nitrile hydratase, or a whole cell or lysed microbial cell biocatalyst comprising said nitrile hydratase.

15. The method of claim 12 which uses an intact microbial cell biocatalyst which optionally may be fresh (i.e., straight from fermentation); stored, e.g., stored as frozen (frozen as wet); or dry such as a lyophilizate or spray-dried form thereof.
